(19) United States
(12) Patent Application Publication
(10) Pub. No.: US 2015/0335276 A1
(43) Pub. Date: Nov. 26, 2015

(54) DEVICE FOR ASSISTING THE VISUALLY IMPAIRED

(71) Applicant: Anthony Chase, Apopka, FL (US)

(72) Inventor: Anthony Chase, Apopka, FL (US)

(21) Appl. No.: 14/283,706

(22) Filed: May 21, 2014

(51) Int. Cl. A61B 5/15

(52) U.S. Cl. CPC A61B 5/150748

(57) ABSTRACT

A device for assisting the visually impaired to collect blood samples for a glucose testing unit is disclosed. The device includes a finger attachment unit having a shape and size that is complementary to a portion of a human finger. An aperture is disposed along a middle section of the finger attachment unit and is in communication with a collection funnel. The collection funnel and the finger engagement unit are arranged at a substantially perpendicular angle to each other, and the collection unit provides an unobstructed pathway through the aperture.

### TECHNICAL FIELD

The present invention relates generally to medical devices, and more particularly to a device for assisting the visually impaired to collect blood samples for glucose testing.

### BACKGROUND

The statements in this section merely provide background information related to the present disclosure and may not constitute prior art.

In the United States alone, nearly 19 million people have been diagnosed with diabetes, and nearly 2 million additional Americans are diagnosed each year. For many of these individuals, particularly those suffering from type 2 diabetes, it is extremely important to constantly monitor glucose levels in the blood, owing to the body's inability to generate sufficient levels of insulin.

The most common form of blood sugar monitoring involves the use of one or more commercially available glucometers, which utilize a test strip containing glucose oxidase, an enzyme that reacts to glucose in the blood. In this regard, it is not uncommon for individuals to use an instrument called a lance to prick their finger(s) several times a day, in order to obtain a blood droplet that can be applied to the test strip for analysis by the glucometer. Based on the output of the glucometer, the user may then perform corrective treatment such as taking a shot of insulin, to ensure the blood sugar levels remain within acceptable limits.

If not controlled, diabetes puts patients at high risk for a host of complications that can affect nearly every organ in the body. For example, diabetes patients suffer increased risks for heart attacks, kidney failure, and nerve damage which can result in leg and foot amputations. In addition to the above, one of the most common side effects of diabetes includes loss of vision. The loss of vision can begin with the development of cataracts, and can eventually lead to glaucoma and blindness. In this regard, diabetes is the leading cause of blindness in adults age 20-74 in the United States.

Owing to the above, many individuals suffering from diabetes and impaired vision find it increasingly difficult to obtain a suitable blood sample, and to then provide the sample to the test strip of a glucometer. For example, without good vision the process of simply aligning the small lancing instrument, which is about the size of a pen, with a finger is difficult. As a result, many users accidentally lance the side of their finger or strike other portions of the hand that are extremely painful. Moreover, once the lance has pierced the skin, the user must expertly place the test strip onto the blood without sliding the strip across the finger. If this occurs, most glucometers will generate an error message and then require the user to draw another blood sample.

In light of the above, it would be beneficial to provide a device for assisting the visually impaired to collect blood samples for a glucose testing unit, without suffering from the above noted drawbacks.

### SUMMARY OF THE INVENTION

The present invention is directed to a device for assisting the visually impaired to collect blood samples for a glucose testing unit. One embodiment of the present invention can include a finger attachment unit having a shape and size that is suitable for engaging a portion of a human finger. The unit can also include an aperture that is in communication with a collection funnel. The collection funnel and the finger engagement unit can be arranged at a substantially perpendicular angle to each other, and the collection unit can provide a pathway for directing blood testing equipment through the aperture and onto a specified portion of a user's finger.

Another embodiment of the present invention can include a finger attachment unit that can be constructed from an elastomeric material in order to stretch and conform to the shape of the user's finger.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of the a device for assisting the visually impaired that is useful for understanding the inventive concepts disclosed herein.

FIG. 2 is a side view of the finger attachment unit of the device for assisting the visually impaired, in accordance with one embodiment of the invention.

FIG. 3 is a side view of the collection funnel of the device for assisting the visually impaired, in accordance with one embodiment of the invention.

FIG. 4 is a perspective view of the device for assisting the visually impaired in operation, and in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

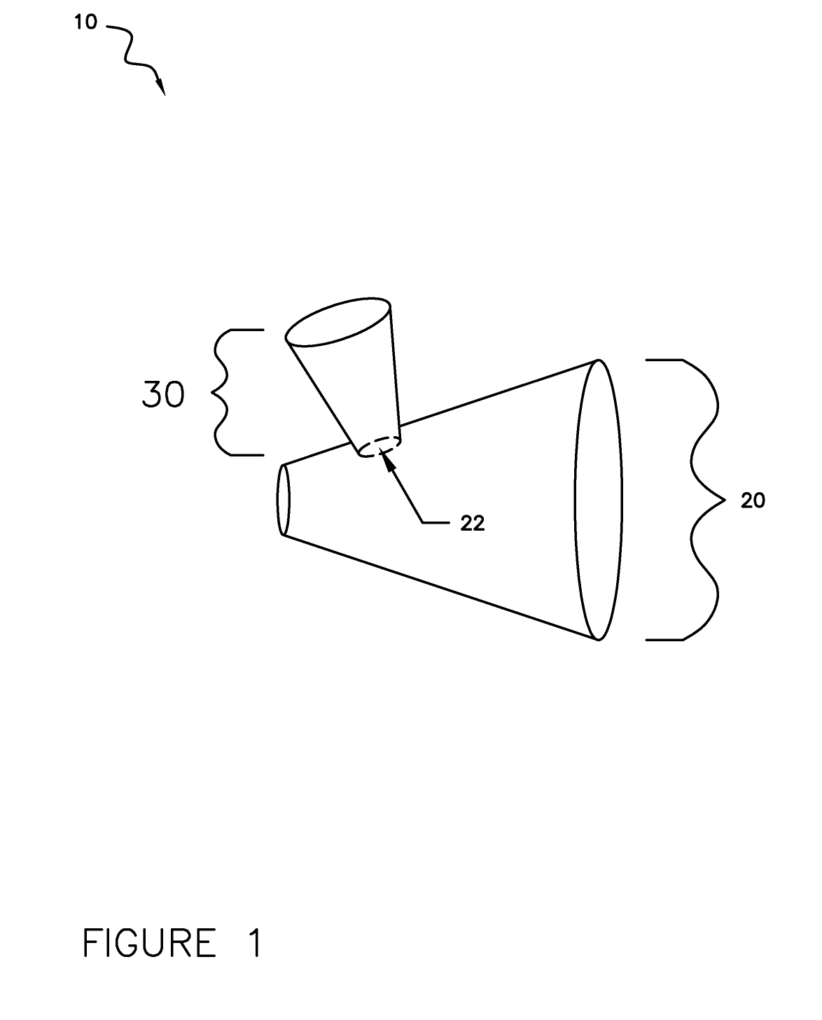

Identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

Although described herein as a device for assisting the visually impaired to collect blood samples for glucose testing, those of skill in the art will recognize that many other uses for the presently claimed invention are possible. Accordingly, the device is not to be construed as limiting to any particular use or industry.

FIGS. 1-3 illustrates one embodiment of a device for assisting the visually impaired to collect blood samples for glucose testing that is useful for understanding the inventive concepts disclosed herein. As shown, the device 10 can include, essentially, a finger attachment unit 20 having a collection funnel 30 secured thereto.

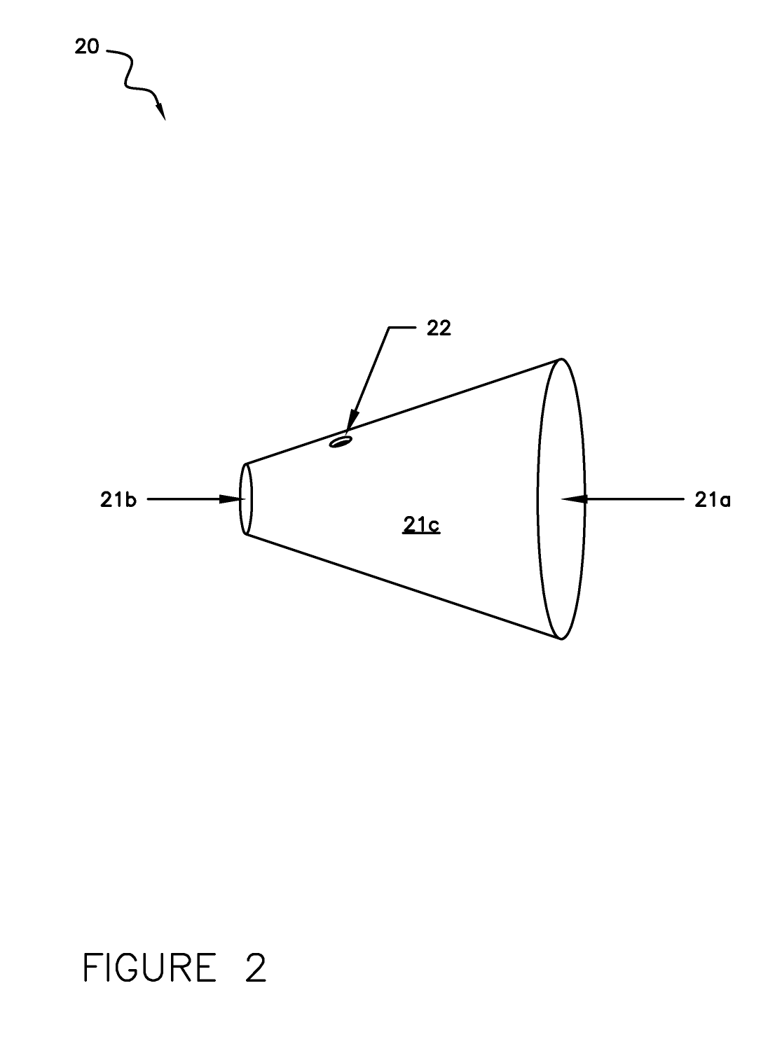

The finger attachment unit 20 can function to receive a human finger and to position the collection funnel at a precise location along the finger, in order to obtain a blood sample. As shown best in FIG. 2, one embodiment of the finger attachment unit can include an elongated hollow and generally frusto-conical shaped main body having a first end 21a, a second end 21b, and a middle section 21c. An aperture 22 is located along the middle section and extends from the outside surface to the inside surface of the main body.

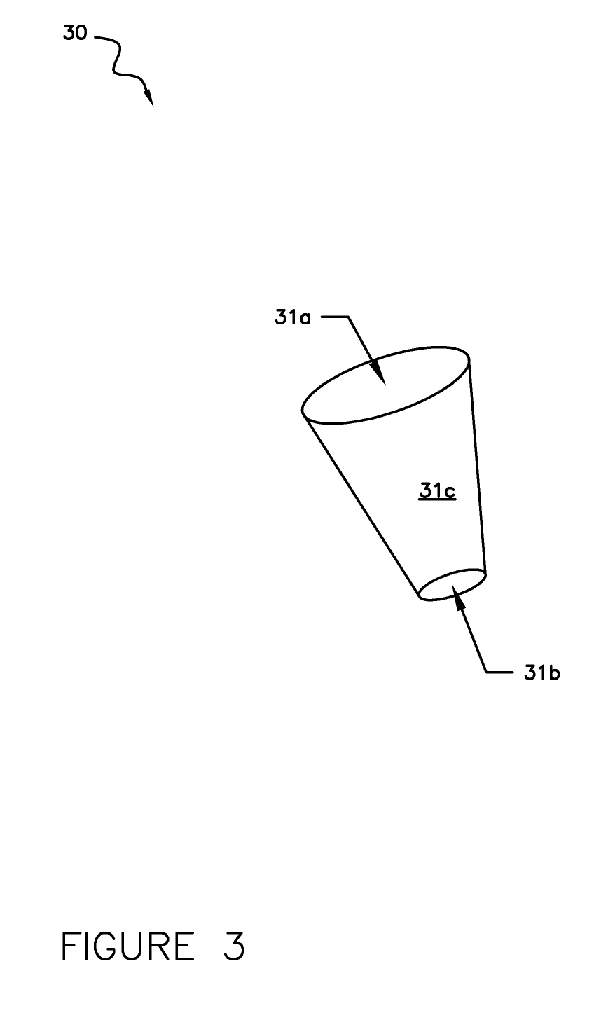

The collection funnel 30 can function to assist the visually impaired and/or those with diminished fine motor skills with the precise alignment of glucose testing instruments onto specific portion of a user's finger. As shown best in FIG. 3, one embodiment of the collection funnel can also include an elongated hollow and generally frusto-conical shaped main body having a large first end 31a, and a narrow second end 31b.

In the preferred embodiment, the collection funnel 30 can be secured onto the finger attachment unit 20 at a generally orthogonal angle, so as to align the open end 31b with the aperture 22. In this regard, the second end of the collection funnel 31b can preferably include a diameter that is approximately the same size as the diameter of the aperture 22.

The finger attachment unit and the collection funnel can preferably be constructed from a single mold of injected plastic, so as to form a single lightweight and inexpensive to manufacture device. However, other embodiments are contemplated wherein each of the attachment unit and the funnel are constructed from other materials such as PVC, composites and metals, for example, and are joined together in either a permanent or non-permanent manner via known manufacturing techniques. For example, each of the finger attachment unit and the collection funnel can include adhesive materials and/or strips of hook and loop material, in order to be repeatedly secured together and subsequently separated (i.e., removably secured together). Moreover, the finger attachment unit 20 can also be constructed from a lightweight elastomeric material such as nylon, for example, which can stretch in order to adapt to the specific shape and size of a user's finger.

While the dimensions of the elements are not critical, one preferred embodiment of the device 10 can include a finger attachment unit having an overall length (i.e., measured from the first end 21a to the second end 21b) of approximately 1.75 inches. The first end 21a can include a diameter of approximately 1 inch, and the second end 21b can include a diameter of approximately 0.75 inches. Likewise, the collection funnel can include an overall length (i.e., measured from the first end 31a to the second end 31b) of approximately 0.5 inches. The first end 31a can include a diameter of approximately 1.25 inches, and each of the aperture 22 and the second end 31b can include a diameter of approximately 0.5 inches.

Although described above with respect to particular shapes, sizes and dimensions, this is for illustrative purposes only, as those of skill in the art will recognize that any number of different shapes and sizes can be utilized herein without deviating from the scope and spirit of the inventive concepts. Accordingly, the device is not to be construed as limiting in any manner.

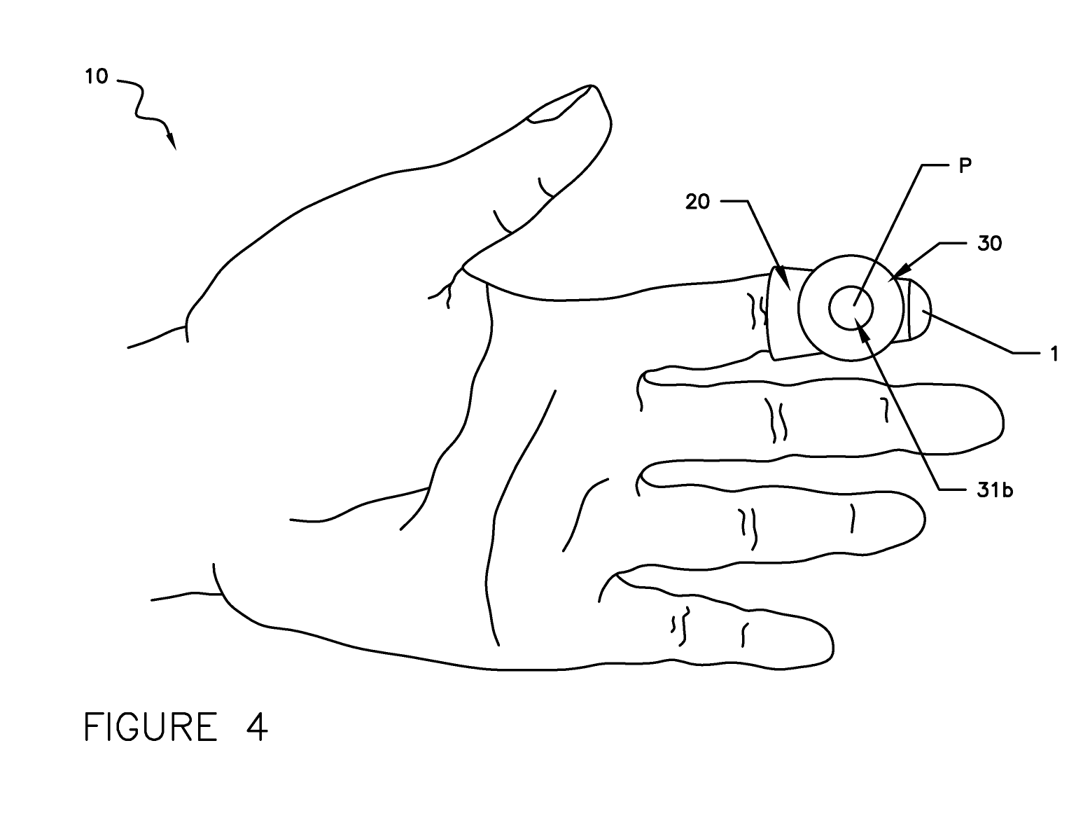

FIG. 4 illustrates one embodiment of the device 10 in operation. As shown, a user can insert any finger 1 into the finger attachment unit 20, in order to align any desirable portion P of the finger beneath the aperture 22 and the second end of the funnel 31b. When so positioned, the shape of the funnel 30 can assist the user to guide the lancing instrument and a test strip or other such device to the desired portion of the finger P.

Moreover, owing to the shape of the attachment unit 20, the users finger can be pressed against the aperture 22 with a force sufficient to allow the blood to pool within the bottom portion of the cone 30, and not run down the sides of the users finger. In this regard, by pooling the blood within the confined space of the funnel 30, the device can ensure that a test strip will receive a sufficient blood sample for an accurate reading by a glucometer.

Accordingly, the above described device provides a simple and inexpensive tool for assisting the visually impaired to collect blood samples for a glucose testing unit.

As described herein, one or more elements of the device 10 can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individual elements such as the finger attachment unit 20 and the collection funnel 30 may be formed together as one continuous element, either through manufacturing processes, such as welding, casting, or molding, or through the use of a singular piece of material milled or machined with the aforementioned components forming identifiable sections thereof.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A device for assisting the visually impaired, said device comprising:
an elongated hollow finger attachment unit that includes a main body having a first end, a second end and a middle section;
an aperture disposed within the middle section of the finger attachment unit; and
a collection funnel that includes a generally frusto-conical shaped main body having a first end and a second end,
said second end of the collection funnel being in communication with the aperture of the finger attachment unit, and said collection funnel and said finger attachment unit including a generally orthogonal relationship with each other.

2. The device of claim 1, wherein the finger attachment unit includes a generally frusto-conical shape.

3. The device of claim 1, wherein the finger attachment unit is constructed from an elastomeric material that is configured to stretch and conform to a shape of a user's finger.

4. The device of claim 1, wherein the finger attachment unit includes a length of approximately 1.75 inches.

5. The device of claim 1, wherein the first end of the finger attachment unit includes a diameter of approximately 1 inch, and the second end of the finger attachment unit includes a diameter of approximately 0.75 inches.

6. The device of claim 1, wherein the collection funnel includes a length of approximately 0.5 inches.

7. The device of claim 1, wherein the first end of the collection funnel includes a diameter of approximately 1.25 inches, and each of the second end of the collection funnel and the aperture include a diameter of approximately 0.5 inches.

8. The device of claim 1, wherein the finger attachment unit and the collection funnel are constructed as a single unitary element.

9. The device of claim 1, wherein the finger attachment unit and the collection funnel are removably secured together.

10. The device of claim 1, wherein the finger attachment unit includes a shape and dimension that is complementary to the shape and dimension of a human finger.
